(19) United States
(12) Patent Application Publication
(10) Pub. No.: US 2011/0264104 A1
(43) Pub. Date: Oct. 27, 2011

(54) HEMODIALYSIS GRAFTS AND METHODS FOR LOCALIZING AND IDENTIFYING THE PLACEMENT OF SAME

(75) Inventor: Joseph Joe Naoum, Houston, TX (US)

(73) Assignee: The Methodist Hospital Research Institute, Houston, TX (US)

(21) Appl. No.: 13/019,186

(22) Filed: Feb. 1, 2011

(30) Foreign Application Priority Data
Aug. 3, 2009 (US) PCT/US2009/052608

(51) Int. Cl. A61F 11/00; A61B 6/00; A61B 17/11

(52) U.S. Cl. 606/108; 606/153; 600/431

(57) ABSTRACT

Disclosed are vascular access devices, implantable dialysis grafts, and systems including them useful for improved access to implanted medical devices. Also disclosed are implantable hemodialysis vascular access graft devices that facilitate easy, accurate and reproducible cannulation or needle entry into the implanted device by magnetically-locating a portion of the graft that includes one or more paramagnetic materials operably defining the physical boundaries of the target cannulation site/entry port.

### CROSS-REFERENCE TO RELATED APPLICATIONS

The present application claims priority from PCT International Patent Application No. PCT/US2009/052608, filed Aug. 3, 2009, which claims priority to U.S. Provisional Patent Application 61/085,678, filed Aug. 1, 2008, the entire contents of each of which is specifically incorporated herein by reference in its entirety.

### BACKGROUND OF THE INVENTION

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### FIELD OF THE INVENTION

The present application relates generally to the construction of hemodialysis and other vascular grafts, and more particularly, to an improved vascular access graft constructions that permit localization and identification of graft placement and/or cannulation site(s) post-implant. In certain embodiments, the use of graft materials that include one or more fixably-positioned magnetic or paramagnetic materials permit the identification and localization of the implanted graft by passing a detector wand (that includes one or more magnets) over the surface of the skin in the region proximate to the implant, thereby aligning a portion of the wand above a first region of interest of the graft.

### BACKGROUND OF THE INVENTION

Vascular diseases affect a significant portion of the world's human population. Bypass surgery, whereby a conduit, either artificial or autologous, is grafted into an existing vessel to circumvent a diseased portion of the vessel or to restore blood flow around a blocked or damaged blood vessel, is one of the most common treatments for such diseases. It is estimated that over 1 million such procedures are performed annually.

The majority of vascular access grafts in use today are as entry sites in patients with end-stage renal disease (ESRD) that requires chronic hemodialysis. While autogenous fistula (e.g., a Brescia-Cimino fistula) is the first choice of arteriovenous (AV) access for hemodialysis, for patients with small veins, or for those patients in whom autogenous grafts do not properly develop into a fistula, heterogeneous arteriovenous graft (AVG) devices, such as synthetic grafts implanted under the skin, represent the only feasible alternative.

AVGs function much like fistulas in many respects, except that an artificial (i.e., synthetic) vessel is used to join the artery and vein. The graft usually is made of a synthetic material, but sometimes chemically treated, sterilized veins from animals or human cadaveric tissues are used. Typical placement sites for AVGs include, without limitation, the forearm, upper arm, neck, and thigh, in either straight or closed loop configurations.

Once surgically positioned, an AVG becomes an artificial conduit that can be used repeatedly for needle placement and blood access during hemodialysis. During dialysis, blood is withdrawn from the graft, passed through a hemodialysis machine, and then returned to the patient through a second needle inserted in the graft.

Because hemodialysis patients undergo repetitive, often painful, large-needle punctures of their skin and underlying tissue numerous times per week to gain entry into surgically implanted AVGs, these implants typically remain patent (and unobstructed) for several months to several years, and must periodically be repaired or replaced. The disadvantages of multiple needle-puncture procedures to access the graft are numerous and well documented. First, hematomas can result from uncontrolled bleeding. Second, grafts can be damaged by the multiple punctures required for routine dialysis. Third, the threat of physical damage to the graft itself, and/or infection at the cannulation site can destroy the integrity of the access graft. Complications with the graft can ultimately lead to poor, inadequate, or incomplete functioning, or, alternatively, to thrombus formation, which, in most cases, results in the need for additional surgical intervention, including, e.g., repair or replacement of the graft, and/or resection of the resultant clots and/or traumatized tissue. Because hemodialysis access grafts are implanted entirely below the skin (to reduce the risk of infection and to provide better comfort to the patient between dialysis treatments) hypodermic needles are used to cannulate the vessel through the skin. During cannulation of the graft, direct punctures of the graft walls are made with such needles. In conventional hemodialysis, two cannulas (typically, e.g., 14- to 16-gauge needles) are placed in the access graft, with one puncture being made in the graft wall in the arterial side and one puncture being made in the venous side.

Conventional dialysis protocols require a patient to undergo a dialysis procedure at least three times a week, with each procedure typically lasting four or more hours. As a result, the number of times an implanted vascular access graft is cannulated in a single month can be a dozen or more. These repeated punctures of the graft material, however, are prone to error and complication. Incorrectly done, the punctures may promote rupture of the graft, pseudoaneurysm formation, and/or the development of organized thrombi within the lumen of the graft. The formation of such blood clots may result not only in multiple graft thromboses, but may eventually lead to graft failure.

Another significant limitation is “finding” the proper position within the subdermally-localized graft to perform the needle sticks. While more superficially-positioned grafts may be readily palpitated through the skin, more deeply-implanted devices provide significant limitations to patients' and medical personnel's accurately localizing the site for needle puncture. Repeatedly missing the graft entirely, or improperly positioning of the needle within the lumen of the graft device are two contraindications, which adversely affect the time the graft remains patent.

Repeated, direct punctures of the graft wall also require compression for hemostasis following the dialysis session. Excessive compression during hemostasis may cause decreased flow within the graft and thrombosis. In addition, there is very little subcutaneous tissue between the surface of the skin and the graft wall reducing the capacity of extra luminal coagulation of the blood within the surrounding tissue and therefore causing reduced hemostasis at the end of the procedure. [As noted above, dialysis grafts may often be difficult to palpate if placed too deeply into the patient's tissue. Accessing deeply placed grafts can be difficult, and significant technical expertise and nursing care is currently required to puncture the grafts. Following dialysis and needle removal, skilled medical personnel are required to hold pressure on the graft puncture site for variable periods of time, which may be as long as one hour post-cannulation. Conversely, if AVGs are implanted too superficially, the graft is more susceptible to infection, which further undermines patency. Therefore, both proper placement of an AVG surgically, and correct identification of its access region following implantation are critical to the usefulness and patency of the device.

Each of these shortcomings represents a significant limitation in the prior art. Devices and methods to overcome one or more of these limitations would provide welcome and necessary improvements over methodologies currently available in the medical arts, and particularly in methods currently available for treating dialysis patients, and managing renal insufficiency and/or kidney failure in affected mammalian populations.

### BRIEF SUMMARY OF THE INVENTION

In view of these and other shortcomings of the prior art, the present invention provides new methods and devices that may advantageously improve access to an implanted graft device (e.g., an arterio-venous (AV) graft device) to facilitate easy, accurate and reproducible entry into the implanted graft using devices such as dialysis needles, cannulas, and the like, which are introduced into the graft via insertion into the skin.

By 1) creating one or more distinct magnetic/paramagnetic site(s) within, upon, or about the actual dialysis graft material, 2) implanting a suitable AV graft made of such material into the patient's body, and 3) by then subsequently utilizing a specially-designed detector “wand” (that contains one or more magnets suitably positioned to identify the implanted graft), the invention advantageously provides one or more of the following: (a) an economic and reliable means of allowing dialysis providers to consistently and accurately access the implanted graft site without extensive expertise; (b) fewer “missed” needle sticks/cannulation errors, (c) less pain and/or discomfort for the patient undergoing the procedure, and (d) reduced opportunity for damaging, destroying, or displacing the implanted graft device due to incorrect insertion of the cannula or improper and/or repeated needle sticks attempting to “hit” the proper insertion site on the subcutaneous graft.

To that end, implementation of the magnet-localizable graft access devices disclosed herein is expected to not only lower the incidence of damage/discomfort to the patient from imprecise punctures, but to also preferably reduce physical damage to the implanted graft material or device itself. By facilitating a more readily-identifiable positioning of the graft and more proper placement of the cannula/needle for puncturing such device, improper punctures, development of graft thrombosis, and secondary complications arising from one or more of these problems (such as bleeding and/or infection) are greatly reduced.

Furthermore, implementation of magnetic-localizable devices (including, for example, magnetic-localizable AV grafts vascular septa, implanted access/entry ports, subcutaneous drug delivery devices, laparoscopic adjustable gastric bands, and the like) will preferably decrease many of the costs associated with such implant procedures, and may also reduce the costs associated with long-term use of the implanted devices, including, for example, long-term hemodialysis, by increasing longevity, patency and usefulness of the graft, lowering the risk of complications and contraindications, and overall decreased patient pain and discomfort associated with access to the grafts, septa, injection ports, access points, and such like that are associated with a variety of implanted medical devices.

Without being bound by theory, it is believed that use of the magnet-localizable implants, access ports, septa, vascular grafts, and the like will promote not only improved patient compliance, but will also make outpatient and in-home dialysis more facile, and more economically feasible for many implanted patients. Moreover, by using a locator (i.e., a detector or a “wand”) that contains at least a first suitably-sized opening defined by the location of one or more magnets therein, to pass over the skin in suspected proximity to the implanted graft, port, septum, or access device, it is now possible to identify and to localize the particular region of the device, or one or more sites in, on, or about the implanted device where puncture/cannulation should optimally occur. This “localizing” ability of a magnet-containing detector can make it easier to find the proper cannulation site without advanced medical training, while minimizing damage to the graft itself. The present invention also in many cases obviates the need for a secondary surgery to reposition a graft that was otherwise too deeply positioned in the tissue to provide ready localization of the cannulation area, septa and/or access ports simply by using conventional methods of palpating (i.e., “feeling for the graft”) through the skin using the medical practioner's hands/fingers to try and locate the area of implant under the skin.

One aspect of the present invention is an implantable vascular access device (as well as its corresponding method of use) that may be readily localized once implanted to a relatively high degree of precision and accuracy within or about at least a first portion of the body of the implanted animal (i.e., patient) without need of direct surgical access or physical intervention to contact the actual implanted device itself.

The invention also preferably provides vascular access devices (as well as their corresponding methods of use) that are localizable through the skin with a high degree of accuracy and/or precision for performing repeated needle sticks or cannulations to at least one ore selected portions of an implanted vascular access graft that is within a region defined by the presence of one or more magnetic materials in, or, or about the graft material itself.

One additional aspect of the invention is an AV access graft, suitable for facilitating or performing dialysis when operably positioned in the circulatory system of the animal, by implantation into the body of such a patient undergoing dialysis, as well as the corresponding method for improving the precision and/or accuracy of identifying the location of deeply-positioned devices, and to facilitate proper cannulation or needle injections into such a deeply-positioned graft device without additional surgical intervention.

Embodiments of the invention also preferably improve the precision of accurate needle/cannula placement into implanted medical devices, including implanted access ports, such as, without limitation, laparoscopic gastric band filling ports, and the like.

Still another aspect of the invention is an implanted device and a method of use that improves the precision for repeated cannulation of such a device when deeply positioned within the body of such a patient, or when positioned within the body of an obese patient, or in circumstances where the physical size, girth, and/or weight of the patient limits or reduces the accuracy of palpating an implanted device through the patient's skin.

The invention preferably also enhances the ability of a patient or medical personnel to properly and accurately identify the placement of implanted medical devices (such as vascular access grafts and such like) immediately following surgical implantation of such devices to ensure proper placement. Should the surgeon identify that the graft is improperly, or imperfectly placed by using the magnetic locator “wand,” the graft can readily be repositioned, and/or modified during the same surgical procedure prior to closing the surgical site. Such method finds particular utility in reducing or obviating the need for one ore more subsequent surgeries to “correct” the placement of the implant. Such methods may thereby also reduce patient discomfort, lessen surgical healing time, reduce hospitalization costs, and lessen/reduce/prevent the chance of a surgical site infection.

The invention preferably also provides a new method and vascular access device of the type described for visualizing at least a first portion of an implanted medical device or graft by conventional medical imaging means, including, for example, x-ray, magnetic resonance imaging, and/or computer-aided tomography (CT).

In still another aspect, the invention preferably minimizes the time to achieve proper cannulation of the graft and to begin the dialysis procedure. Using an external magnet to identify and localize the position of an implanted device that includes a paramagnetic or ferromagnetic material greatly reduces the pain experienced by patients during incorrect cannulation, and greatly facilitates increased patency of the graft, septum, access port, or such like after surgical implantation into the body of the animal.

In certain aspects of the invention, the generally tubular shaped graft may be fabricated from one or more materials that include polytetrafluoroethylene (PTFE, Teflon® Gore-Tex®, Gore Viabahn®, Gore Propaten®, etc.), expanded polytetrafluoroethylene (ePTFE, Gore Intering®, etc.), polyester, polyurethane, nylon, polyethylene terephthalate (Dacron®), and the like, or any combination of the foregoing, or alternatively, a processed blood vessel derived from living or deceased animals, including, without limitation, human or other mammalian donors or cadaver-harvested tissues.

Alternatively, vascular grafts of the present invention can be fashioned in flat sheet forms, conventionally referred to as vascular or cardiovascular “patches” that are used to replace only a portion of the circumference of a vein or artery. The scope of the present invention includes both tubular vascular grafts and flat sheet vascular patches, and the use of the term vascular grafts herein encompasses both tubular and flat-sheet forms.

The graft material will also be manufactured preferably to include at least one component that has paramagnetic or ferromagnetic properties (i.e., a material that is attracted to a magnet), such that following implantation, the graft device may be localized or oriented within the patient's body by passing a magnet over the surface of the patient's skin in proximity to the graft site, thereby allowing the magnet to at least substantially, and preferably entirely, align itself with the graft, essentially marking the optimal site for needle access or puncture of the graft during cannulation.

In an aspect of this invention, the lumen of the artificial vascular graft has a cross-sectional area that is substantially equivalent to a cross-sectional area of a lumen of a vessel to which the tubular element is grafted. The size of the graft, the diameter of its lumen, and the thickness of the material forming the walls of the graft itself may be fabricated in any suitable dimension(s), as may be warranted by its particular medical application, or as may be deemed necessary for correct placement of the implant device, and dependent upon the particular use of the graft, using information that is known to those of ordinary skill in the art of graft/access port device fabrication and such like.

However, in certain generalized aspects of the invention, the cross-sectional area of the lumen of a conventional AV-suitable graft is typically on the order of from about 1 mm² to about 400 mm²; alternatively from about 3 mm² to about 300 mm²; or more preferably from about 5 mm² to about 200 mm² or even more preferably still, from about 7 mm² to about 150 mm², depending upon the function and specific placement of the device within the body of the patient into which it is implanted.

Conventional AV graft devices typically include a length of from about 1 or 2 cm to about 80 or 90 cm; preferably from about 3 or 4 cm to about 60 or 70 cm; more preferably, from about 5 or 6 cm to about 40 or 50 cm, or even more preferably still, from about 7 or 8 cm to about 20 or 30 cm for most human applications (depending of course, upon the specific function and placement of the device within the body of the patient into which it is implanted), although additional graft lengths, including all integer lengths within the aforementioned ranges, are also specifically contemplated to fall within the scope of the present disclosure.

Typical internal diameters of graft implants suitable for use in the practice of the present invention include those conventionally fashioned, as well as those commercially available, and those known to persons of ordinary skill in the medical arts. For example, the internal diameter of AV-suitable graft materials is typically on the order of from about 1 or 2 mm to about 15 to 20 mm; preferably from about 3 or 4 mm to about 12 to 15 mm; and more preferably, from about 5 or 6 mm to about 8 to 11 mm in most conventional human application, although additional diameters, both larger and smaller than the specified sizes, as well as all integer diameters within the aforementioned ranges, are also specifically contemplated to fall within the scope of the present disclosure.

In one aspect, the invention provides a vascular access graft for connecting an artery to a vein, or more than one artery to more than one vein. In an overall and general sense, the device includes a tubular graft of biocompatible material for conducting fluid, the tubular graft anastomosed to the artery at a first end and anastomosed to the vein at a second end; a first septum in the tubular graft that is in fluid communication with the venous side of the graft; a second septum in the tubular graft that is in fluid communication with the arterial side of the graft; and at least first and second paramagnetic rings disposed essentially circumferentially around said tubular graft, wherein the first paramagnetic ring substantially defines the border of the first septum and is in substantial proximity thereto, and the second paramagnetic ring substantially defines the border of the second septum, and is in substantially proximity to the second septum, wherein the prosthetic device is entirely subcutaneous and is capable of being cannulated by a needle disposed through the port chamber.

Preferably, the at least one arterial-side tubular septum and the at least one venous side tubular septum are each connected at opposite ends to the graft such that a continuous lumen is formed. In certain embodiments, the at least one arterial-side tubular septum and/or the at least one venous-side tubular septum are disposed in axial alignment with the tubular graft, and preferably, wherein both septa are disposed substantially along a longitudinal axis of the tubular graft.

In illustrative embodiments, the vascular access device further includes a first cannulating means disposed on an arterial side of the conducting means and a second cannulating means disposed on a venous side of the conducting means. Preferably, the tubular element includes polytetrafluoroethylene (PTFE) expanded polytetrafluoroethylene (ePTFE), polyurethane, polyester, or another suitable biocompatible material as described herein, or any combination thereof.

Preferably, the tubular port chamber is attached to the graft at opposite ends such that a substantially continuous lumen is formed, and the tubular graft is anastomosed in a substantially end-to-side fashion to a first artery at one end and is anastomosed in a substantially end-to-side fashion to a first vein at an opposite end.

In certain embodiments, the at least one septum on the at least one arterial-side tubular port chamber and on the at least one venous-side tubular port chamber have sidewalls that extend beyond the graft such that the septum further includes at least a first ring including at least a first paramagnetic or magnet-localizable material disposed substantially uniformly around, and/or substantially circumferentially-defining the opening in the device's port chamber, septum, or access port.

In some embodiments, the at least one tubular port chamber may be spliced into, or constructed substantially within the graft device prior to, during, or following manufacture of the device; or alternatively, may be introduced into the graft device immediately prior to, or during the surgery in which the device is implanted into the recipient patient.

The access ports, septa, and port chambers of the disclosed devices may include, consist essentially of, or alternatively, consist of, a first substantially inert, biocompatible material that is adapted to be penetrated by a needle, cannula, or catheter system, to facilitate transfer of fluids into or out of the access device through the port, septum, or port chamber. In certain embodiments, the material may be a self-sealing insert, to permit repeated needle punctures of the device without destroying the integrity of the septum or port including the material. The use of self-sealing port and septal materials in the formation of implantable vascular access devices is well known in the art and exemplified in one or more of the patents specifically incorporated herein by express reference thereto.

In the case of hemodialysis, the transfer of fluids through the device may be the removal of blood from within a first vessel of a patient implanted with the device, and/or reintroduction of blood within a second vessel of the patient. Alternatively, the transfer of fluids through the device may involve the introduction or removal of fluid (such as e.g., saline or a radio-opaque material), or both, into or from a laparoscopic gastric band to properly control the volume of fluid in the band following laparoscopic bariatric surgery. Alternatively, the transfer of fluids through the device may involve the introduction of one or more drugs, small molecules, dyes, diagnostic reagents, or such like via an implanted drug delivery device, including, for example, insulin delivery devices for use in the treatment of diabetic patients. The transfer of fluids through the device may alternatively involve the removal of serum, blood, plasma, lymphatic, sciatic, ascetic or other bodily fluid, such as for the quantitative or qualitative assessment of one or more compounds in such fluids. In one embodiment, the fluid may be returned to the body through the same connection following assessment.

In another aspect the invention provides an implantable vascular access device that generally includes a) a tubular graft of biocompatible material for conducting fluid, the tubular graft anastomosed to an artery at least at a first end; b) a tubular, biocompatible, port chamber that is in fluid communication with the graft, wherein the port chamber has at least one septum defined therein, the septum being formed substantially by at least a first hole defined within the port chamber, and being covered by a biocompatible penetrable material; and c) at least a first paramagnetic material disposed substantially around the port chamber, where the paramagnetic material substantially defines the border of the septum. Preferably, the septum is fabricated of a material that is essentially self-sealing.

Preferably, the paramagnetic material used in formation of the disclosed medical devices will include, consist essentially of, or consist of, iron, steel, cobalt, nickel, a ceramic material, surgical-grade steel, or an alloy or combination thereof.

Alternatively, the material used in the formation of the disclosed medical devices may include, consist essentially of, or consist of, a superparamagnetic material, including for example, superparamagnetic metal oxide nanoparticles (e.g., superparamagnetic iron oxide nanoparticles [SPIOs] (see e.g., Ji et al., 2007, which is specifically incorporated herein in its entirety by express reference thereto.)

The invention also provides an implantable dialysis graft, that generally includes a substantially tubular graft of at least a first biocompatible material for conducting fluid within the lumen of said graft, and extending substantially between at least a first artery and at least a first vein; and, at least two tubular port chambers in fluid communication with the tubular graft, the port chamber having at least a first septum defined therein, the first septum formed by a first hole substantially defined in at least a first portion of the port chamber, wherein the hole is covered by a biocompatible penetrable material and defined by at least a first paramagnetic ring portion disposed substantially around a first portion of the port chamber; wherein the dialysis graft is implanted entirely subcutaneously, and is adapted to be cannulated by a needle disposed through the first hole in the port chamber, wherein the port chamber is localized by passing a magnet along the skin of the patient into which the graft is implanted, such that the location of the port chamber is facilitated by the alignment of the magnet substantially above the first paramagnetic ring on the first portion of the port chamber.

Preferably, the device is fabricated to permit an effective flow rate for hemodialysis, or an effective delivery rate for addition or removal of a fluid from within the graft.

The invention also provides systems and methods for using the disclosed devices in a variety of medical indications. In one illustrative embodiment, the invention provides a system for identifying and more accurately localizing the positioning of an implanted vascular graft or device. This method generally employs the use of a system that includes (a) a magnetically-localizable vascular device as disclosed herein; and (b) an external magnet that is capable of localizing and detecting the implanted vascular graft including a paramagnetic material, when the magnet is placed in proximity to the skin of a patient into which the graft or device has been implanted, preferably in proximity to the graft site.

While any nominal band thickness ordinarily used in fabrication of medical devices is contemplated to be useful in preparation of grafts in accordance with the present invention, band thickness in the range from about 0.002 inch to about 0.01 inch is preferred. Exemplary surgical stainless steel grades contemplated to be useful in the manufacture of exemplary devices according to the invention include, without limitation, surgical steel commonly graded as 410-, 416-, 420-, 430-, or 440-series stainless steel.

The wound rings (i.e., bands) comprised of a magnetic material are fabricated circumferentially around the outer wall of a standard AV graft and the band chaffing may be positioned on the graft starting approximately 5 to 10 mm from each end and spaced along the graft at various intervals. The graft may be of any conventional size, but those of about 1 cm to about 10 cm are preferable, with those on the order of about 3 cm to about 8 cm being more preferable still. Examples of such grafts include, without limitation, non-tapered grafts (including those, for example, from about 5 mm to about 8 mm in length for dialysis); tapered grafts (including those, for example, having a combination of about 4-5 mm, 4-6 mm, 4-7 mm, and 4-8 mm, as well as those of about 5-6, 5-7, and 5-8 mm; and those, for example, of 6-7, or 6-8 mm also being preferable; standard-wall stretch PTFE, thin wall PTFE, multi-layer patch grafts (including, for example, those having elastomeric fluoropolymer or accu-seal-like components); polyurethane urea grafts; carbon-coated grafts; Dacron grafts; arterial homografts; venous homografts; bovine or other animal-derived grafts; and umbilical vein grafts.

The number of metal rings spaced circumferentially around the graft may be of any practical number, it is envisioned that grafts comprising from 2 to about 8 or 10 bands (preferably placed equidistant along a substantial portion of the long axis of the graft device, or equidistant with larger spacing between each end of the graft and the closest ring to that end) will be preferable for most applications. As such, the distances between each magnetically-attractive band, will preferably be on the order of from about 10 or 20 mm to about 60 or 80 mm (as measured center-to-center), with bands spaced at intervals from about 30 or 40 mm to about 50 or 60 mm being most preferred.

Optionally, the devices of the present invention may include one or more final or outer wraps. Exemplary constructs include graft/band assemblies that are substantially spirally-wrapped along a portion or the entire length of the graft device with one or more layers of the ePTFE tape (including, without limitation ½″ wide ePTFE tape commercially available in the medical device arts) with approximately 5% to 20% overlapping, with 10 to 15% overlapping being preferable in most embodiments of the invention. The final outer wrap may then be secured to the device using conventional methods, including, without limitation, by bonding or sintering onto the graft surface. Alternatively, the layer(s) of wrapping on the graft device may be secured to the outer surface of the device by suturing, or a biocompatible sealant, glue, or adhesive (including, without limitation, BioGlue® [CryoLife, Kennesaw, Ga., USA; bovine serum albumen and glutaraldehyde] and such like).

Exemplary magnets for use with the disclosed access devices, grafts, and vascular access ports will preferably include, consist essentially of, or alternatively consist of, a ceramic, lanthanoid, paramagnetic, ferrimagnetic, or ferromagnetic material, including, but not limited to, those that include aluminum, boron, cobalt, copper, iron, neodymium, nickel, samarium, titanium, or a combination or alloy thereof, including, but not limited to commercially-available permanent alloy magnets, such as, without limitation, NdFeB, AlNi, AlCoMax, AlNiCo, TiConAl, and the like.

In certain aspects, the magnet may be at least substantially, or preferably entirely, circular or toroidal in shape, and may have one or more holes at least substantially, or preferably entirely, centrally located in the magnet such that when the magnet is aligned substantially directly, or preferably directly, over the implanted device, a needle may be passed through the magnet (while still in place on the patient's skin) directly through the tissue and into the septum, port, or graft access at a preferred point of cannulation. The term “substantially,” as used herein in connection with a shape, preferably refers to a shape that is within about 20 percent, preferably within about 10 percent, and in some embodiments within about 5 percent, of the normal parameters for that shape. With reference to a circle, for example, “substantially” could mean that every point on the circumference is preferably within about 20 percent of the diameter of the circular shape. Alternatively, the magnet may be used to mark a position on the skin where the needle stick is desired, or alternatively may be used to align the point of needle puncture, but removed prior to the actual cannulation of the access site itself.

Exemplary magnets for use in these methods include, but are not limited to, those that are about 1 cm to about 5 cm in diameter, and those that are at least substantially “hockey puck-shaped,” “donut-shaped,” toroidally-shaped, cylindrical, or such like, to facilitate proper needle or cannula placement upon localization of the implanted magnet-localizable graft device.

In the practice of the invention, the magnetic detector wand used in localizing the implanted graft device may be of any suitably durable material, such as for example, one or more plastic materials, or the like. In one embodiment, the wand may be fabricated of a non-magnetic plastic material into which rare earth magnets are inserted with a center-to-center designed to coincide with the spacing of the metal rings displaced around the circumference of the implanted graft device. In an illustrative embodiment, the wand includes two ⅜″ diameter rare-earth magnets operably positioned with a center-to-center spacing of about 40 mm. As shown in the accompanying figures, the side of the wand opposite the handle is preferably open to allow easy access to between the magnets with the hypodermic needle to facilitate ready access to the graft through the patient's skin.

While any suitably sized magnets may be used, exemplary magnets include Neodymium Iron Boron (NdFeB) cylindrically shaped (e.g., “button”) magnets. In certain embodiments, the magnets may be coated with one or more protective layers to facilitate maximum protection and durability. In one example, nickel-copper-nickel triple-layer coatings commonly employed in the magnet fabrication industry may be used to coat the magnets contained in the device-localizing wand to facilitate magnet durability and longevity. Exemplary magnets suitable for use in the disclosed devices include, without limitation, grade N45 magnets with a typical maximum remanence of 13200 gauss. Magnets employed in exemplary detection wands of the present invention preferably are magnetized through their entire thickness, and preferably have a pulling force of at least approximately 10 to 20 lb strength (with pulling forces of at least approximately 12 to 16 lb being particularly preferable for certain embodiments).

The magnetic properties of certain neodymium magnets contemplated to be useful in the practice of the invention, are found in the following table:

TABLE 1
MAGNETIC PROPERTIES OF EXEMPLARY
NEODYMIUM MAGNETS

| | | Max.Energy | Coercive | Intrinsic | Maximum |
|---|---|---|---|---|---|
| | | Product | Force | Coercive | Working |
| | Remanence | (BH)max | Hcb | Force | Temp. |
| Grade | (BrmT) | (MGO) | (KOe) | Hci (KOe) | (° C./° F.) |
| N35 | 1170-1210 | 33-36 | ≧10.9 | ≧12 | 80/176 |
| N40 | 1250-1280 | 38-41 | ≧10.5 | ≧12 | 80/176 |
| N50 | 1400-1450 | 48-51 | ≧10.0 | ≧11 | 80/176 |

Additionally, the present invention provides a vascular access graft, an implantable vascular access device, an implantable dialysis graft, or a system as disclosed herein for use in therapy, and in particular, in the therapy of diabetes, obesity, and/or renal therapy.

Use of a vascular access graft, an implantable vascular access device, or an implantable dialysis graft as provided herein is also contemplated in the manufacture of a medicament, therapeutic kit, or medical device for the treatment of one or more diseases, disorders, dysfunctions, or trauma, including, for example, the treatment of diabetes, obesity, and/or renal dysfunction, including the delivery of one or more drugs through an implanted access graft of port, the hemodialysis of a patient in acute or chronic kidney disease or renal failure, or the proper filing of solution in an implanted laparoscopic gastric band following bariatric surgery.

A further aspect of this invention relates to a method of performing hemodialysis on a patient using an artificial vascular graft of this invention. The graft in this aspect is implanted under the skin of the patient with one end of it grafted into an artery and the other end grafted into a vein whereby fluidic continuity is established from the artery through the lumen of the tubular element and into the vein. The lumen of the graft is connected to a hemodialysis filtration unit such that blood can be diverted from the lumen into the hemodialysis filtration unit, filtered, and then returned into the lumen. Localization and identification of graft placement post-implantation may be facilitated by the presence of one or more magnetic materials included within or in close proximity to, the implanted graft.

In another embodiment the invention provides magnetically-localizable multi-layer grafts that generally includes a first tubular structure having a first porosity; a second tubular structure having a different porosity than the first porosity, wherein the second tubular structure is disposed externally about the first tubular structure; and a self-sealing material is interposed between the first and second tubular structures, wherein the self-sealing material is selected from the group consisting of sheet, film, yarn, thread, mono-filament wrap, multi-filament wrap, tube, solvent-spun elastomeric fibers, helically-wound tape, and combinations thereof. In such embodiments, the multilayer grafts are generally comprised of expanded PTFE or other suitable material such that the first porosity is greater than the second porosity.

Exemplary self-sealing materials include, without limitation, thermoplastic elastomers, silicones, silicone rubbers, synthetic rubbers, polyurethanes, polyethers, polyesters, polyamides, fluoropolymers and combinations thereof. In certain embodiments, the grafts of the present invention may be augmented with one or more pre-sintered fluoropolymer bead wraps.

Artificial vascular grafts of this invention may also be used in place of any current by-pass or shunting graft, either natural or artificial, in any application. Thus, they may be used for, without limitation, arterial by-pass, both of the cardiac variety and that used to treat peripheral arterial disease (PAD), or for drug delivery, or for implanted filing ports, such as those used in laparoscopic adjustable gastric band (i.e., Lap-Band®) surgery. In addition, the vascular access grafts of the present invention may also be used to replace an absent, defective, diseased, occluded or partially-occluded vessel, or otherwise traumatically damaged vessel of the lymph or circulatory systems, including for example, traumatically damaged limb arteries and such like.

Examples of expanded PTFE suitable for use in the practice of the invention include, without limitation, PTFE compositions as set forth in U.S. Pat. Nos. 6,620,190 and 6,719,783; 7,462,675; and 7,510,571 (each of which is specifically incorporated herein in its entirety by express reference thereto).

Vascular devices in connection with the present invention also include those devices that comprise one or more coatings, including for examples, those comprising one or more silane copolymers or antimicrobial coatings including, without limitation, those as described in U.S. Pat. Nos. 7,029,755; 7,151,139; 7,179,849; 7,547,445; and 7,563,734 (each of which is specifically incorporated herein in its entirety by express reference thereto). Drug-eluting vascular devices including, without limitation, those described in U.S. Pat. Nos. 7,384,660; 7,413,781; 7,468,210; 7,527,632; and 7,563,278 (each of which is specifically incorporated herein in its entirety by express reference thereto) may also be employed in the practice of the invention.

Hemodialysis grafts are widely used in medicine, as exemplified by U.S. Pat. Nos. 3,826,257; 4,549,879; 4,753,640; 5,713,859; 5,716,395; 6,146,414; 6,156,016; 6,461,321; 6,582,409; 6,585,762; 7,025,741; 7,452,374; and 7,566,316; (each of which is specifically incorporated herein in its entirety by express reference thereto).

Self-sealing arteriovenous grafts (including, without limitation, those described in U.S. Pat. Nos. 5,192,310; 5,628,782; 5,700,287; 5,716,395; 5,910,168; 6,102,884; 6,428,571; 6,926,735; 7,083,644; 7,223,257; 7,244,271; and 7,452,374 (each of which is specifically incorporated herein in its entirety by express reference thereto).

Grafts according to the present invention may further comprise one or more valves for facilitating dialysis. Exemplary valve devices include, without limitation, those described in U.S. Pat. Nos. 7,211,074 and 7,540,859 (each of which is specifically incorporated herein in its entirety by express reference thereto).

AV graft devices in accordance with the present invention may also optionally comprise one or more subcutaneous ports to facilitate enhanced graft access. Exemplary catheter systems include, without limitation, those described in U.S. Pat. Nos. 7,566,316 and 7,008,412 (each of which is specifically incorporated herein in its entirety by express reference thereto).

Methods for inserting AV grafts are known to those of ordinary skill in the art, and include, without limitation, methods as set forth in U.S. Pat. No. 5,306,240 (specifically incorporated herein in its entirety by express reference thereto). In an overall and general sense, to place a graft in accordance with the invention, an incision is typically made in a target vein, the venous end of the graft is introduced into the interior of the vein and placed a predetermined distance downstream from the venotomy, and the graft is sealingly secured to the vein wall. In exemplary embodiments, a venous anastomosis is achieved by (1) making an incision in the wall of a preselected target vein; (2) inserting the venous end of an inventive graft through the incision into the vein such that the first end passes to a point downstream of the incision; (3) securing the graft to the vein; and (4) anastomosing the arterial end to a preselected target artery.

While important aspects of the present invention are directed to use of the disclosed methods and devices in human medicine, the inventor also contemplates that the invention will be of benefit for enhanced localization and improved placement of implanted devices in a variety of animals, including, for example, those under veterinary care.

### BRIEF DESCRIPTION OF THE DRAWINGS

For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The following drawings form part of the present specification and are included to demonstrate certain aspects of the present invention. The invention may be better understood by reference to the following description taken in conjunction with the accompanying drawings, in which like reference numerals identify like elements, and in which:

FIG. 1A and FIG. 1B illustrate plan views of an exemplary AV graft flexed (FIG. 1A) and native (FIG. 1B) and magnetic detector wand in accordance with one embodiment of the invention. Shown are the metal bands along the length of the graft conduit, and the spacing of the magnets on the detector wand that align substantially with the placement of adjacent rings along the conduit to localize the device when implanted under the skin of the patient. In FIG. 1B, an exemplary cannulation device is shown piercing the graft in the designated cannulation region located between pairs of adjacent rings (drawing not to scale).

FIG. 2A, FIG. 2B, FIG. 2C, and FIG. 2D illustrate plan, oblique, and side views, respectively, of an exemplary AV graft in accordance with one embodiment of the invention, and the relative placement of the magnetic detector wand above the graft device. As in FIG. 1A and FIG. 1B, shown are the metal bands along the length of the graft, and the spacing of the magnets on the detector wand that align substantially with the placement of adjacent rings along the conduit to localize the device when implanted under the skin of the patient. In FIG. 2C, an exemplary cannulation device is shown piercing the graft in the designated cannulation region located between pairs of adjacent rings (drawing not to scale). FIG. 2D shows optimal machining of the magnet containment portions of the detector device to facilitate an angulated guide for assisting in the proper placement angle for a needle to access the cannulation site or septum;

FIG. 3A shows a cross-sectional view of a exemplary graft showing the graft wall (1), the internal lumen of the device, and a metal band/ring (3) fixably-adhered to the outer surface of the device using, for example, a bio-compatible bonding adhesive (2). Alternatively, as described in the legend to FIG. 4, the metal bands may be sutured into position on the ablumenal side of the tubular graft, or held in place by ePTFE wraps, fibers, wires, etc.;

FIG. 3B shows a plan top view of an exemplary AV graft in accordance with the invention showing the relative position of the plurality of metal band/rings circumferentially disposed substantially uniformly along the longitudinal axis of the graft; shown with the graft is an exemplary magnetic wand that has magnets placed at corresponding center-to-center distances to facilitate detection of the implanted device through the patient's skin;

FIG. 4 shows a flexible sleeve design in accordance with another embodiment of the invention that can be used to fit over or substantially around an autogenous venous conduit or other biological homograft to facilitate proper localization/identification of the graft once implanted. In such embodiments, a sleeve that contains a plurality of metal bands circumferentially disposed substantially uniformly along its long axis may be slipped over the autogenous venous conduit or homograft to facilitate localization. The metal rings may be bonded to the sleeve in any conventional manner, including, e.g., by a PTFE overwrap, by physical suturing, or by fixation using a biocompatible adhesive;

FIG. 5 shows a side plan view of another embodiment of the invention in which a magnetic detection wand may be fabricated to detect a conventional port-a-cath device. In such embodiments, a metal ring may be bonded or adhered to the distal end of the cannulation port (i.e., opposite to the end of the port in fluid connection to the lumen of the catheter);

FIG. 6 shows an illustrative schematic of retrofitting an existing device port with a metal band or ring to facilitate detection by a corresponding magnetic detection wand passed over the patient's skin at the site of device implant. If the body or structure of the port itself is made of stainless steel or an otherwise magnetic material, then all that is required for localization of the port is a substantially correspondingly-shaped detector wand that contains a magnetic ring appropriately sized to facilitate detection of the implanted port;

FIG. 7 shows an illustrative implantation scheme for the implant shown in FIG. 6. As illustrated, the portacath (alternatively, Porta-Cath or Poart-A-Cath) device is implanted under the skin of the patient (shown here in an arm), with the port connected to a catheter that has been anastomosed to a vessel inside the patient's arm. The cannulation site on the port has been fitted with a metal band or ring that essentially defines the preferred needle entry site. By passing the magnetic wand over the patient's skin at the site of device implant, the precise localization of the implanted port may be achieved. As shown, a needle or cannula is this suitably positioned within the area defined by the detection wand to facilitate more accurate cannulation of the port;

FIG. 8A and FIG. 8B show the surgical site, and tunneling, respectively, of an illustrative embodiment AV access graft of the present invention into a first surgical site of the body of a porcine mammal;

FIG. 9A, FIG. 9B, FIG. 9C, FIG. 9E, and FIG. 9F are a sequential series of photographs taken from a video record made of the in situ experiment described in Example 4, showing the sliding of a detector wand along the surface of the skin of the animal proximate to the region where the AV graft device was previously surgically implanted. (The surgical site incision has been left open merely for convenience, and to demonstrate visually the internal placement of the device in the cadaver animal). The detector wand is moved around until the magnetic field attracts the wand to the embedded surgical stainless steel rings placed along the longitudinal axis of the implant. In FIG. 9E, a second operator now prepares to cannulate the graft in the interval defined by the magnets on the detector wand; and

FIG. 10A, FIG. 10B, FIG. 10C, FIG. 10D, and FIG. 10E are a continuing sequential series of photographs taken from a video record made of the in situ experiment described in Example 4, that demonstrate a second operator puncturing the needle (FIG. 10A) through the skin and into the lumen of the device at the first cannulation site, which is defined by the embedded rings within the device. In FIG. 10D and FIG. 10E, proper placement of the needle into the cannulation site of the graft is demonstrated by the first operator's introduction of saline through a first end of the graft—saline can be seen exiting the hub of the needle, indicating that the tip of the needle has properly entered the cannulation site.

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

Hemodialysis

A significant number of individuals suffer from decreased kidney function. When the kidney function is depreciated enough (usually to approximately 10 to 20% of normal level), an individual must either receive a kidney transplant or begin undergoing kidney dialysis procedures. Survival of non-transplanted patients with chronic renal failure depends on optimal regular performance of dialysis. If this is not possible, for example, because of vascular access dysfunction or failure, chronic renal failure can lead to a rapid clinical deterioration. Unless the situation is quickly remedied, the patient will die.

Hemodialysis is routinely practiced method for treating patients suffering from renal failure. In 2003, it was estimated that approximately 40,000 new patients begin hemodialysis each year in the United States. Since then, the number has risen annually at a rate of about 10 percent. Hemodialysis instruments (i.e., “artificial kidney,” “dialysis machine,” “dialyzer,” etc.) serve to remove life-threatening chemicals, such as toxins, cellular breakdown products, and the like, from the bloodstream, when the patient's kidneys can no longer effectively remove such chemicals on their own.

In hemodialysis, a patient's blood is cleansed by passing it through a dialyzer-essentially an artificial kidney machine—that consists of two chambers separated by a thin membrane. Blood passes through the chamber on one side of the membrane and dialysis fluid circulates on the other. Waste materials in the blood pass through the membrane into the dialysis fluid, which is discarded, and the cleansed blood is re-introduced into the patient's bloodstream.

In order to perform hemodialysis, repeated access must be obtained to the patient's circulatory system; blood flow rates of about 100 to 500 ml/min to and from the body are typically required for optimum dialysis conditions. Blood from veins is inadequate to meet these flow requirements, and repeated puncture of a large artery is not feasible.

To overcome these limitations, vascular access is therefore the method typically used to access the bloodstream of a hemodialysis patient. Because hemodialysis involves removal of blood from the body, routing it to an external device in which the blood is cleansed, and then returning the cleansed blood back to the patient, a critical feature of hemodialysis is easy and routine access to the patient's circulatory system.

The most common forms of gaining access to the circulatory system is by vascular access. The types of vascular access include (a) formation of an AV fistula; (b) placement of a temporary central venous catheter; and (c) placement of a prosthetic arteriovenous access graft (AVG). Nearly two-thirds of all hemodialysis patients in the United States have an implanted AVG to facilitate cardiovascular access and permit repeated hemodialysis.

Vascular Access Grafts

Vascular access grafts in general, and hemodialysis arteriovenous grafts in specific, are well known in the medical arts. Approximately 100,000 vascular access procedures are performed yearly in the United States. Maintaining a patent access to the circulatory system is of paramount importance in hemodialysis patients. However, because many patients have poor native vessels, artificial graft material in many instances represent the mode of choice for chronic and repeated vascular access.

A variety of conventional types and placements of hemodialysis grafts are well known to those of ordinary skill in the art. Regardless of where the graft device is placed within the body of the patient, the function of the graft is to facilitate withdrawal of blood from the patient for treating the blood in the dialysis instrument.

Vascular grafts may be of biological or synthetic (i.e., “artificial”) origin. Examples of biological grafts include autograft and allograft vessels. An autograft is a vessel that is taken from one site in a patient's body, and then subsequently implanted into another site within the patient's body. For instance, in peripheral vascular surgery, the most common graft includes the long saphenous vein in which the valves have been surgically removed with an intraluminal cutting valvutome. Alternatively, in allograft surgery, a blood vessel is taken from another animal of the same or different (xenograft) species, and used for implantation into the patient. For human patients, vascular allografts may often be obtained from human cadavers, organ and/or tissue donors, or non-human mammals such as primates, ungulates, ruminants, and such like.

Synthetic or artificial grafts are conventionally made of non-biological materials, including PTFE, ePTFE, or other suitable materials as described herein. Presently, results with synthetic grafts below the inguinal ligament are considered inferior to biological (venous) grafts. However, when suitable venous graft material is not available, a graft fabricated from a synthetic material may be used. Use of a synthetic graft also results in a shorter operation, and spares veins for future procedures.

Recent dialysis advances involve the implanting of dialysis access ports beneath the skin. These ports generally contain a chamber plugged with a self-sealing material, such as rubberized silicone, with a synthetic catheter extending out from within the chamber. The port is placed under the skin and the catheter is surgically implanted into a vein. A second port is similarly implanted beneath the skin and its catheter is surgically implanted into another portion of the vein. One port may then be used to remove blood for dialysis while the other port is used to return the cleansed blood back to the body.

AV Graft (AVG) Placement and Localization

Patients requiring dialysis must be fitted with an access capable of collecting and returning blood to and from the filtering device. Often this access site takes the form of an AVG attached between an artery and vein in the patient's body. Essentially an AVG is a length of plastic tube, usually made of porous polytetrafluoroethylene (PTFE), which is surgically placed under the skin, fluidly connecting an artery and a vein. Once the graft is implanted, a dialysis machine can be fluidly connected to the patient's circulatory system by inserting needles into the graft and connecting the needles to the dialysis machine with appropriate tubing. The four most common grafts include a forearm loop graft, an upper arm straight graft, an axillary loop graft and a thigh graft.

While a forearm graft (preferably in the non-dominant arm) is preferred to placement of an upper arm graft, if neither upper extremity graft is deemed suitable, placement in the thigh may be indicated. For upper arm grafts, a common straight graft is typically used, which may begin at the distal radial artery and connect to the cephalic, median cubital or basilic vein in or near the antecubital fossa distal to the brachial access feeding artery and connect to the proximal basilic or axillary vein.

Similarly, a common loop or thigh graft may connect to the cephalic, median cubital or basilic veins, the proximal brachial artery to axillary vein, or the superficial femoral artery to the greater saphenous vein. Other sites may be selected if the patient has a history of previously used graft sites. Typical hemodialysis access grafts have relatively high systolic velocities (e.g., about 100 to about 400 cm/s), high diastolic velocity (e.g., about 60 to about 200 cm/s), high flow, and low resistance.

Regardless of the site of implantation, in order to access the graft for blood filtration, needles must be inserted at either end of the graft, whether it is natural or synthetic. Poor access technique can lead to serious damage to the graft material, infection, hematoma and improper dialysis. A deep graft placement is often used to decrease the chance of infection, but can be especially difficult to access.

In important embodiments, the present invention provides prosthetic vascular grafts that offer improved localization upon implantation, thereby improving the process of cannulating the graft and significantly reducing the risk of blood loss or graft compromise when the graft is improperly punctured by a dialysis needle.

Cannulation of the grafts of the present invention may be accomplished by conventional methods widely employed in hemodialysis. In an overall and general sense, cannulation typically involves inserting the point of a suitable needle or cannula into an upper surface of the graft with the bevel of the needle facing upwards, that is, away from the surface of the graft. Preferably, the point of the needle is through a first septal surface of the graft to intersect the longitudinal axis of the graft. It is desirable that the needle be aligned with the graft so that the longitudinal axis of the needle and the longitudinal axis of the vascular graft lay in a common plane during cannulation. To facilitate such cannula entry, the magnetic detector wand may be fabricated as shown in the accompanying figures to provide an angulated guide for directing the proper placement angle of the needle. In the practice of the invention, it is desirable that each needle used to cannulate the graft is oriented at an angle of between about 40 and about 60 degrees, with an entry angle of about 45 degrees (with respect to the longitudinal axis of the graft) being particularly preferred. In placing the needles into the graft, care should always be taken so as not to damage the opposite or lower surface of the graft, or to puncture the graft completely across its entire width such that the opening to the needle has passed completely through the proximal surface of the graft, the lumen, and the distal surface of the graft coming to rest somewhere in the tissue infra to the implanted graft device. In such circumstances, cannulation must be repeated so that the needle opening come to rest within the lumen of the graft such that fluid removal may occur from the lumen and into the cannula.

AVG Patency

The most common problem related graft failure is a condition known as intimal hyperplasia, which can occur when the higher pressure/volume of the arterial flow crosses the boundary from the relatively non-compliant graft to the more compliant outflow vein at the venous anastomosis. The resultant intimal hyperplasia in the vein adjacent to the anastomosis leads to progressive stenosis and eventually premature clotting and graft occlusion. Repairing an AVG occlusion is typically facilitated by one of several techniques: surgical thrombectomy, administration of one or more thrombolytic drugs, or mechanical de-clotting through an interventional approach known as percutaneous mechanical thrombectomy (PMT). PMT techniques include a variety of different approaches including, without limitation, suction thrombectomy, balloon thrombectomy, clot maceration and mechanical thrombectomy. The goal of each of these therapies is the preservation of vascular access. In almost all cases, however, any technique that is used to de-clot the graft also requires angioplasty of the venous anastomotic stenosis in order to reestablish normal flow.

Construction of AVGs

Any suitable material available to those of ordinary skill in the art may be used alone or in combination to prepare one or more layers of the disclosed vascular grafts. Preferably, materials that may be used include, but are not limited to, silicones; silicone rubbers; synthetic rubbers; polyethers; polyesters; polyolefins; modified polyolefins such as, for example, halogenated polyolefins that include, but are not limited to, fluorinated polyolefins; polyamides; FEP; PFA; polyurethanes; segmented-polyurethanes; segmented polyether-polyurethanes; polyurethaneurea; silicone-polyurethane copolymers; and, any analogs, homologs, congeners, derivatives, salts, and any combination thereof.

The disclosed vascular access grafts may also include, at least in part, any material suitable for incorporation into one or more layers of the graft to provide flexibility, durability, structural integrity or rigidity, or to permit the lumen of the graft to remain open. Examples of suitable materials for use in the manufacture of the disclosed vascular grafts include, but are not limited to, metals, metalloids, alloys, polymers and any combination(s) thereof. Examples of suitable metals and metal alloys include, but are not limited to, ELASTINITE® (Guidant Corporation, St. Paul, Minn., USA), NITINOL® (Nitinol Devices and Components, Fremont, Calif., USA), stainless steel, tantalum, tantalum-based alloys, nickel-titanium alloy, platinum, platinum-based alloys such as, for example, platinum-iridium alloys, iridium, gold, magnesium, titanium, titanium-based alloys, zirconium-based alloys, alloys including, without limitation, cobalt and chromium (ELGILOY®, Elgiloy Specialty Metals, Inc., Elgin, Ill., USA). Examples of suitable polymers include, but are not limited to, segmented-polyurethanes and other segmented or block copolymers with similar structural properties.

Examples of suitable segmented-polyurethanes include, but are not limited to, polyether urethane ureas, polyether urethanes and polyester urethanes. While segmented polyurethanes are highly effective base polymers for use in the present invention, other segmented or block copolymers with similar structural properties may also be used. Examples of other suitable segmented or block copolymers include, but are not limited to, polyester-polyethers, polyesters, polyether-polyamides, polyamides, styrene-isoprenes, styrene butadienes, thermoplastic polyolefins, styrene-saturated olefins, polyester-polyester, ethylene-vinyl acetate, ethylene-ethyl acrylate, ionomers, thermoplastic polydienes. Reinforced rubbers may be used where the reinforcement serves the same purpose as the hard block in the segmented copolymer. In one embodiment, the graft may include one or more polyesters such as, for example, Dacron® (DuPont, Inc., Wilmington, Del.) or Hytrel®. (DuPont., Inc.). In another embodiment, the graft may include one or more polyamides such as, for example, Nylon®. (DuPont, Inc.).

In another embodiment, the graft material may include a suitable metal or metal alloy. In one example, the metal or metal alloy is a ferromagnetic composition. In another example, the graft may further optionally include one or more low-ferromagnetic or non-ferromagnetic compositions, or a combination thereof. In another example, the graft may include one or more stainless steel, surgical-grade steel, metal, metalloid, or ferromagnetic components disposed on, within, or between one or more layers of the vascular access graft. Alternatively, one or more cannulation access ports may be included within the graft, and these access ports may be fabricated to include one or more magnetic materials suitably positioned within, along, or in proximity to the access port to provide more precise localization of the access port through the application of a magnetic to the surface of the patient's skin such that the magnet is attracted to the ferromagnetic component(s) of the implanted graft and consequently aligns itself with, and preferably over, the ferromagnetic component(s) associated with the graft.

The structural dimensions of the disclosed vascular access devices and cannulation ports can vary within the range of dimensions known to be useful to one of skill in the art. In some embodiments, the graft components can be uniform in thickness or variable in thickness throughout the layer. In other embodiments, a wall thickness for a first inner layer component and a second inner layer component can independently range from about 0.1 millimeter to about 1 millimeter, or any thousandth of a millimeter within the range.

The inner lumen of the disclosed grafts may be designed to promote endothelialization for prevention or inhibition of thrombus formation. As described above, the inner lumen can be porous or rough to promote endothelialization; at least partially coated with an antiplatelet, anticoagulant, antifibrin, or antithrombin to prevent or inhibit thrombus formation, or alternatively, may be treated in ways known to those of skill in the art to prevent and/or inhibit thrombus formation. Other ways to treat inner lumen include, but are not limited to, designing the inner lumen to act as a scaffolding for host cells that secrete polypeptides that are antithrombogenic and modifying the surface of the inner lumen with, for example, polyethylene glycol.

The use of collagen as a material for fabrication of biodegradable medical devices has been widespread in the literature, as illustrated by U.S. Pat. Nos. 6,726,923, 6,323,184, 6,206,931, 6,162,247, and 4,164,559; each of which is specifically incorporated herein in its entirety by express reference thereto.

The vascular access grafts and medical implants of the present invention may also be at least partially coated with a biocompatible material, biocompatible gel or matrix, or one or more drugs or molecules. Exemplary coatings for the disclosed medical implants include, but are not limited to, an antiplatelet, anticoagulant, antifibrin, antithrombin, or a combination thereof, to prevent or inhibit thrombus formation, or an antibiotic, antimicrobial, or anti-inflammatory compound or composition. The devices of the present invention may also include one or more metals or materials that are attracted to a magnetic field.

The grafts in accordance with the present invention are desirably fabricated from one or more biocompatible elastomeric polymer(s) or other biocompatible non-elastomeric materials, foamed polymers and such like. Such graft devices may optionally be coated with one or more drugs or biotherapeutic agents. Exemplary drugs or biotherapeutic agents include, without limitation, hemostatic agents, antibiotics, anti-tumorigenic agents, cell cycle-regulating agents, and thromboresistant compounds such as chondroitin sulfate, dermatan sulfate, heparin, heparin sulfate, hirudin, keratin sulfate, and lytic agents such as urokinase, streptokinase, and the like, and any combination thereof.

Examples of antiplatelet, anticoagulant, antifibrin and antithrombin drugs include, but are not limited to, albumin, gelatin, glycoproteins, heparin, hirudin, recombinant hirudin, argatroban, forskolin, vapiprost, prostacyclin, dextran, D-Phe-Pro-Arg-chloromethylketone (i.e., synthetic antithrombin), dipyridamole, platelet receptor antagonist glycoprotein IIb/IIIa antibodies such as 7E-3B, thrombin inhibitors such as Hirulog (bivalirudin) (e.g., ANGIOMAX®, The Medicines Co., Parsippany, N.J., USA), and any analogs, homologs, congeners, derivatives, salts and/or combinations thereof.

The use of implanted devices including one or more access ports, septa, or injection sites is well known in the art. Recent advances in the field include the development of self-sealing graft inserts, and septa that can be repeatedly cannulated without leaking or deteriorating. Likewise, various mechanical access ports and valves are also known in the art for surgical implantation, and identification and localization of each of these type devices, particularly when deeply-implanted, or difficult to palpate manually through the skin, are contemplated to be improved and facilitated by the methods and devices of the present invention.

It will also be appreciated that the vascular graft devices and access ports of the present invention may have uses other than for dialysis. Such uses include situations where patients require frequent vascular injections or infusions of therapeutic fluids. Other uses include situations where a patient may require constant, or periodic but frequent, monitoring of blood gases or frequent drawing of blood, such as patients relying on in-home cardiac support systems. In such cases, the readily-localizable graft or device may be implanted, and the magnetic detection device utilized to assist with more precisely localizing the implanted graft and/or port by passing the magnet over the surface of the patient's skin in the area of the graft until the magneto-attractive compounds included within the implanted device are recognized by the localized magnetic field, and placement of the magnet over the device is then achieved.

Use of the magnet-localizable devices and implants of the present invention typically greatly reduces the number of “missed” needle sticks, and generally facilitates greater accuracy in identifying the implanted device into which needle cannulation is desired.

The present invention also provides for use of the devices and systems of the present in therapy. Use of the disclosed vascular access grafts and kits including them in the manufacture of a medicament or medical device for treating renal insufficiency, or for providing kidney dialysis in a patient is also provided.

The present invention may also advantageously provide therapeutic and diagnostic kits useful in the treatment and monitoring of renal failure, and for determining kidney function. Other aspects of the invention include methods for identifying the placement of an implanted vascular access device, as well as methods for localizing such placement through the use of a paramagnetic material included within the graft device itself in conjunction with a small external magnet that is passed over the surface of the patient's skin to more precisely and rapidly locate the graft, or one or more access ports within the graft.

Commercial Kits

The present invention also provides kits and other commercial-ready adaptations of the disclosed devices to facilitate improved hemodialysis in a patient implanted with one or more AVGs as disclosed herein.

For example, such kits may comprise, in a suitable container, an implantable device, either alone or in combination with one or more devices or components required for surgical implantation of the AVG. The kits of the present invention may also optionally include one or more magnetic wand devices for localizing and identifying the particular portion of the subdermally implanted device. Such localizing wand may comprise one or more magnets, optimally spaced to assist medical practitioners in precisely localizing the implanted AVG. The kits of the present invention may further optionally comprise one or more instruction(s) or protocol(s) detailing the recommended protocol for implanting (and/or subsequently accessing) the disclosed. Such kits may be prepared for convenient commercial packaging, sale, use, and transport. Such packaging means may incorporate the use of clear or opaque plastics, as well as hard, or flexible packaging.

Exemplary Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of clarity, the following specific terms are defined below:

All integers and sub-ranges within a given range of measurement (e.g., length, concentration, diameter, etc.) are also specifically considered to fall within the scope of the present teaching. For example, where a particular range of graft length is given, for example, “between about 4 and about 12 cm” or “from about 0.001 inches to about 10 inches” or “within the range of from 0.001% to 50%,” etc., it is specifically intended that all intermediate sub-ranges are explicitly included within the scope of the present invention. Likewise, all intermediate integers within a stated concentration range or sub-range are also explicitly encompassed by the present teaching. Therefore, it is understood that recitation of a graft length that falls within the range of “between about 4 and about 12 cm” (inter alia, e.g., 5 cm, 6 cm, 7 cm, 8 cm, 9 cm, 10 cm, and 11 cm) implicitly fall within the scope of the present teaching and the subject matter claimed herein. Likewise, the present specification encompasses both open-ended (e.g., “at least 1%,” “at least 1.5%,” “less than about 2%,” “not more than 5 percent”etc.), as well as all closed-ended sub-ranges within a stated numerical range (e.g., the sub-ranges “between about 0.001 inch and about 1.0 inches” or “between about 0.01 mm and about 10 mm” each implicitly falls within the stated numerical range.

As used herein, the term “ablumenal” refers to the outer side of the graft surface, i.e., the surface that is the side opposite to the “lumenal,” or blood-contacting side of the graft.

As used herein, the term “comprising” and its cognates are used in their inclusive sense; that is, equivalent to the term “including” and its corresponding cognates.

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

As used herein, the term “patient” (also interchangeably referred to as “recipient” “host” or “subject”) refers to any host that can serve as a recipient for one or more of the vascular access devices as discussed herein. In certain aspects, the recipient will be a vertebrate animal, which is intended to denote any animal species (and preferably, a mammalian species such as a human being). In certain embodiments, a “patient” refers to any animal host, including but not limited to, human and non-human primates, avians, reptiles, amphibians, bovines, canines, caprines, cavines, corvines, epines, equines, felines, hircines, lapines, leporines, lupines, murines, ovines, porcines, racines, vulpines, and the like, including, without limitation, domesticated livestock, herding or migratory animals or birds, exotics or zoological specimens, as well as companion animals, pets, and any animal under the care of a veterinary practitioner.

In accordance with long standing patent law convention, the words “a” and “an” when used in this application, including the claims, denotes “one or more.”

The term “e.g.,” as used herein, is used merely by way of example, without any intended limitation, and should not be construed as referring only those items explicitly enumerated in the specification.

### EXAMPLES

The following examples are included to demonstrate illustrative embodiments of the invention. It should be appreciated by those of ordinary skill in the art that the techniques disclosed in the examples that follow represent techniques discovered to function well in the practice of the invention, and thus can be considered to constitute preferred modes for its practice. However, those of ordinary skill in the art should, in light of the present disclosure, appreciate that many changes can be made in the specific embodiments which are disclosed and still obtain a like or similar result without departing from the spirit and scope of the invention.

### Example 1

### Construction of a Magnetically-Localizable Graft Device

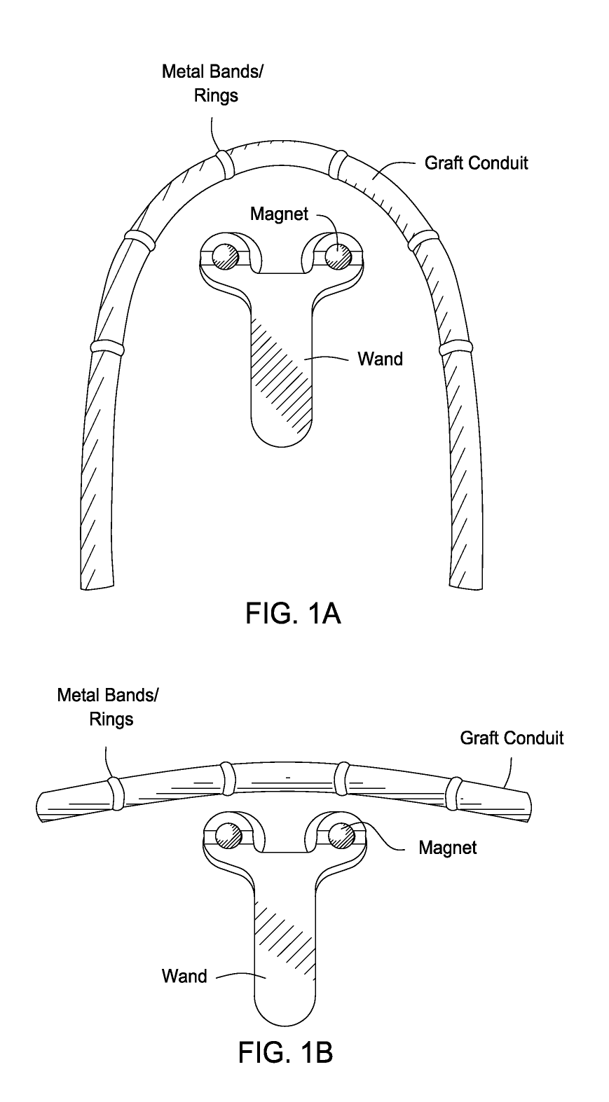

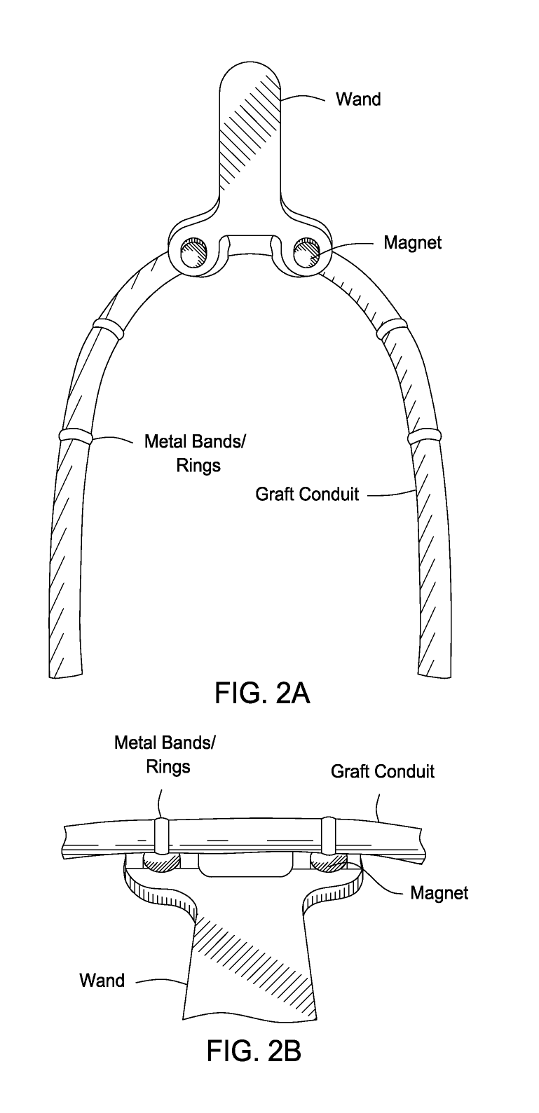

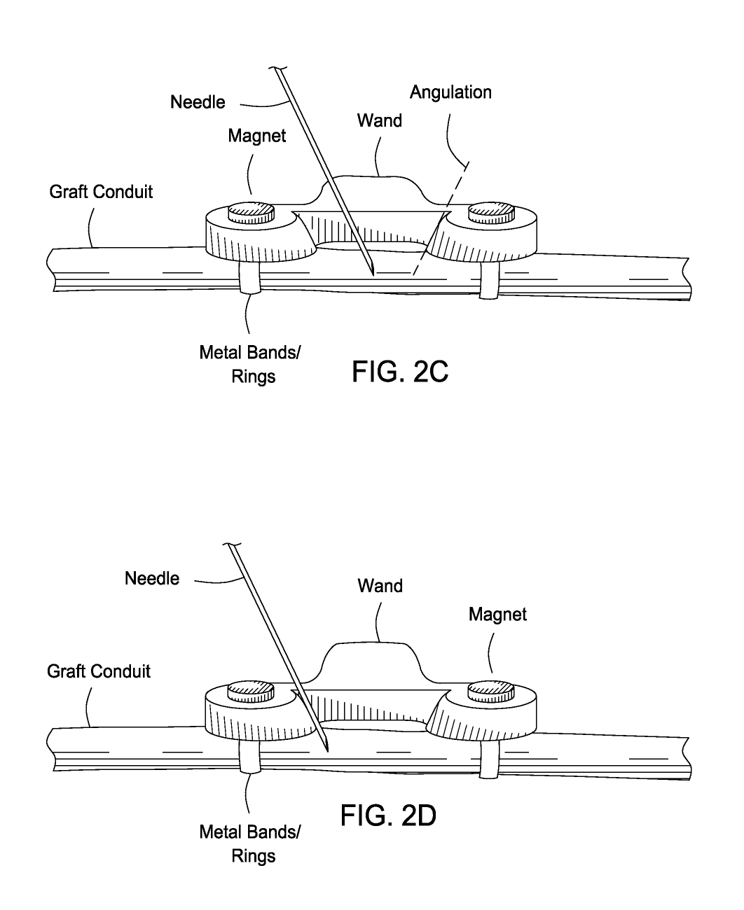

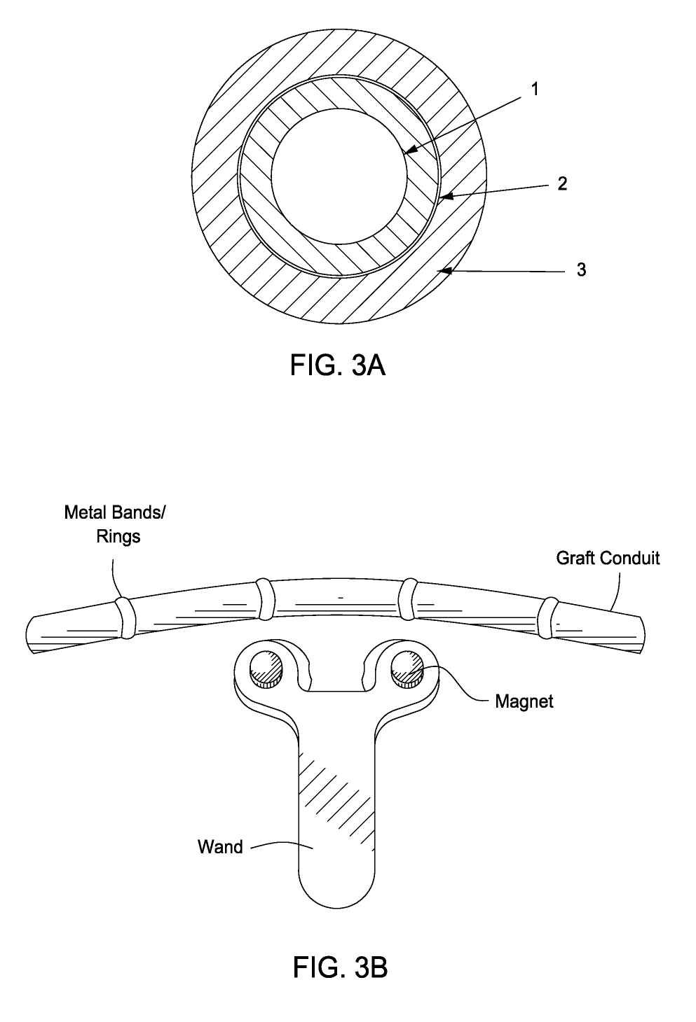

A prototype magnetically localizable graft device was constructed that had six magnetic bands spaced at 4-cm intervals (center to center) along the graft length. The bands were held in position, and chaffed using ePTFE with approximately 1 to 1½ mm overlap of the edges. The assembled graft contained a single layer of ePTFE tape that was spiral wound onto the graft with minimal overlap of the layer (see, e.g., FIG. 1A, FIG. 1B, FIG. 2A, FIG. 2B, FIG. 2C, FIG. 2D, FIG. 3A, and FIG. 3B).

The bands were constructed from 0.006-inch thick Series 420 surgical stainless steel. The chaffing utilized was 0.0025-inch thick ePTFE cut into strips of approximately 5 to 6 mm. The top wrap was fabricated from 0.0025-inch thick×½″ wide ePTFE tape. Bands contained 3-mm wide (1 to 5 mm) strips of Series 420 surgical stainless steel wound to produce a double layer ring of material with approximately 10% overlap.

The magnetic detector wand was made of a non-magnetic plastic material with ⅜″ diameter rare earth magnets having a center-to-center spacing of approximately 40 mm. The side of the wand opposite the handle was open to allow easy access to between the magnets with the hypodermic needle during cannulation of the septum (see e.g., FIG. 1A-FIG. 3B).

The magnet specifications were as follows: Grade N45, BrMax: 13200 gauss; 0.375-inch diameter×0.375-inch length neodymium-iron-boron (NdFeB) cylinder magnet having a nickel-copper-nickel triple layer coating and magnetized through its thickness to an approximate pulling force of about 14 lbs.

### Example 2

### Construction of a Magnetically-Localizable Autogenous Venous Conduit

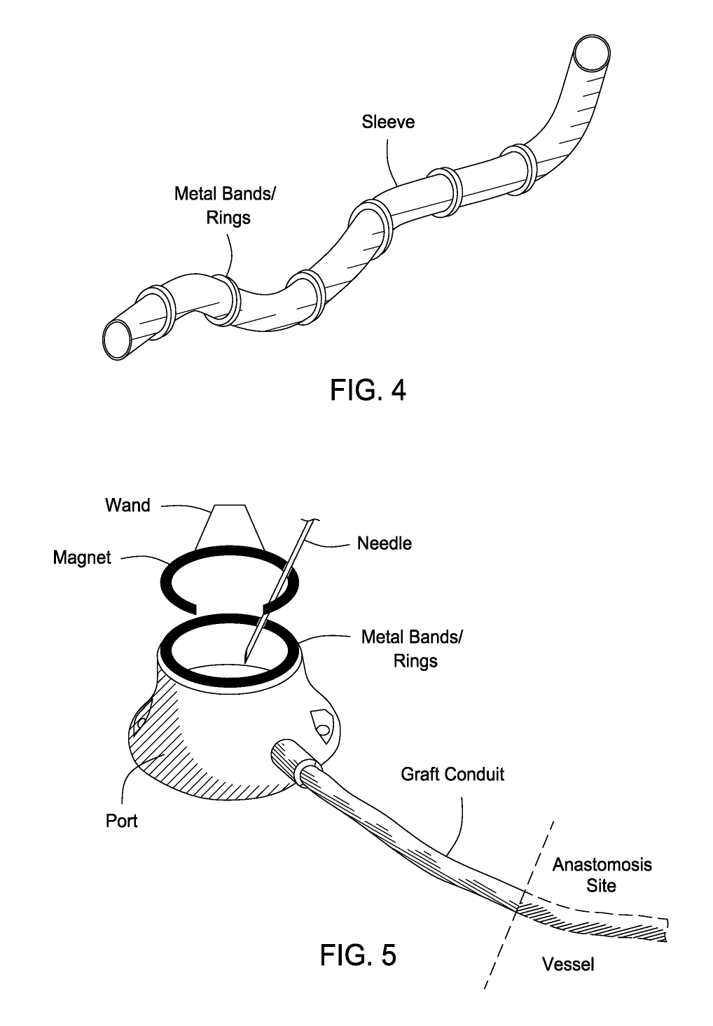

In certain situations, it may be desirable to employ an autogenous venous conduit (AVC) or a biological homograft in place of a synthetic AVG device. In such instances, the devices and localization methods of the present invention may readily be adapted for use in detecting and localizing the position of such biological grafts in situ. As shown in FIG. 4, a flexible sleeve is readily fabricated that includes along its length a plurality of metal bands similar to those utilized in the synthetic graft devices described above. The sleeve is fabricated out of a suitable material such as ePTFE, and then slipped over the AVC and secured to the vessel by suturing, bonding, or application of one or more biocompatible surgical adhesives.

A magnetic detection wand (fabricated such that its magnets correspond in relative dimension to the placement of the metal bands along the length of the graft) is then placed above the skin of the patient, and moved until the attraction of the magnets positions the wand over the implanted magnetically-detectable sheath-encased/AVC hybrid.

### Example 3

### Construction of a Detector Wand For Port-a-Cath Devices

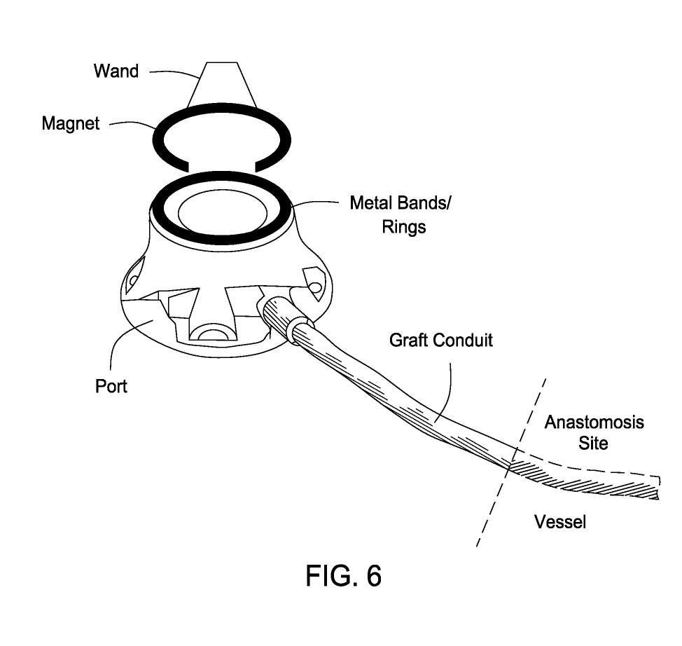

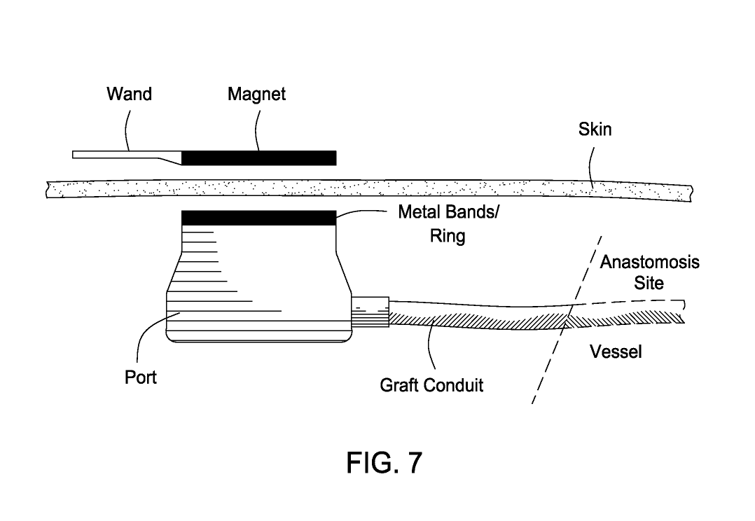

In FIG. 5 and FIG. 6, a conventional port-a-cath device is shown that has been adapted for use in the present localization methods by fabrication of a magnetic detector wand to the corresponding dimensions of the port. In cases where the conventional port is fabricated of non-magnetic materials, the device may be readily modified to contain a metal ring fixably attached to, and defining the outer circumference of, the port. A correspondingly-shaped detector wand is then fabricated to contain one or more magnets operably positioned to facilitate detection of the port in situ. Alternatively, if the port is already fabricated from a magnetic material (such as surgical stainless steel, for example) the device may be detected simply by fabrication of an appropriately sized detector magnet and/or wand that may be wanded over the patient's skin until the embedded port device is localized by the magnet's attraction to the metal port or to a metal ring fabricated to encircle the port as shown.

### Example 4

### Localization of Graft Implant in an Animal Model

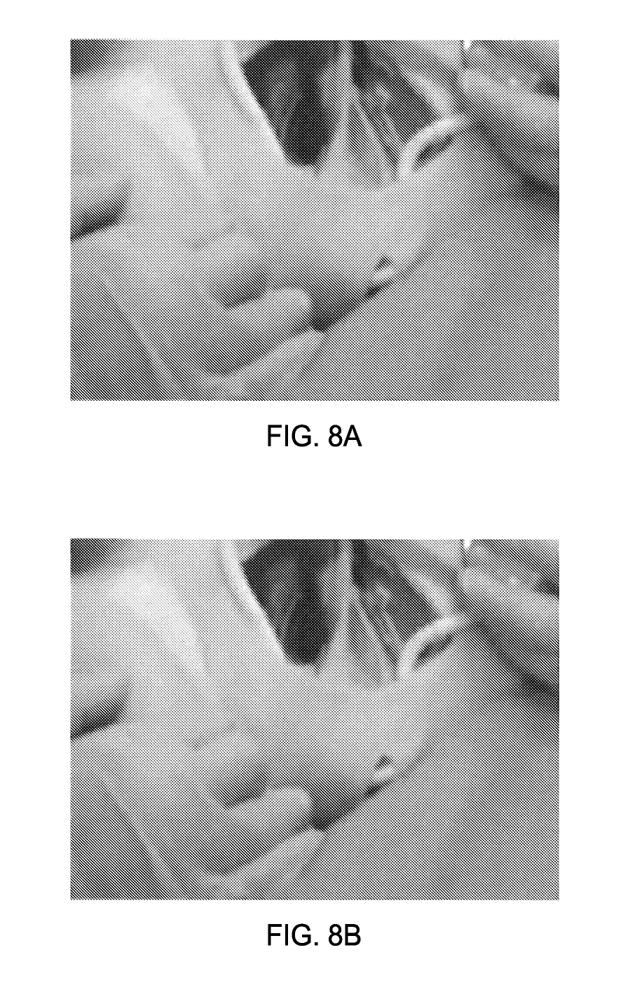

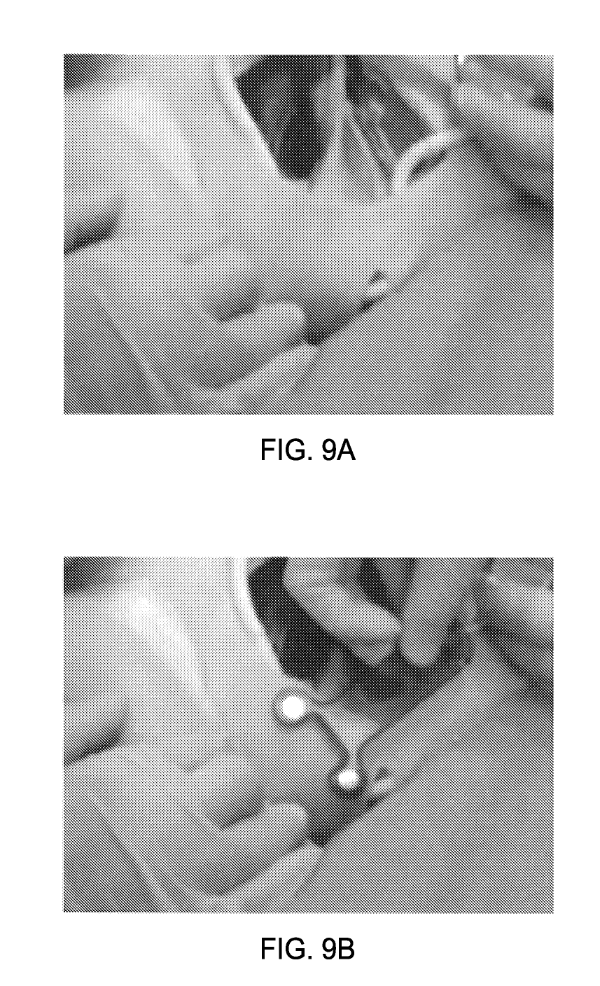

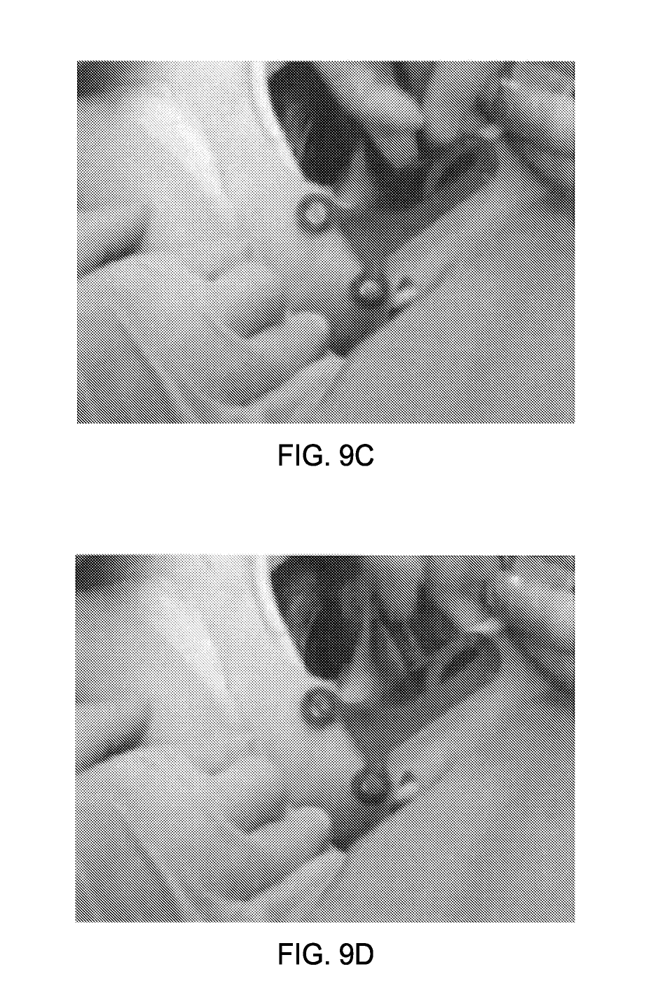

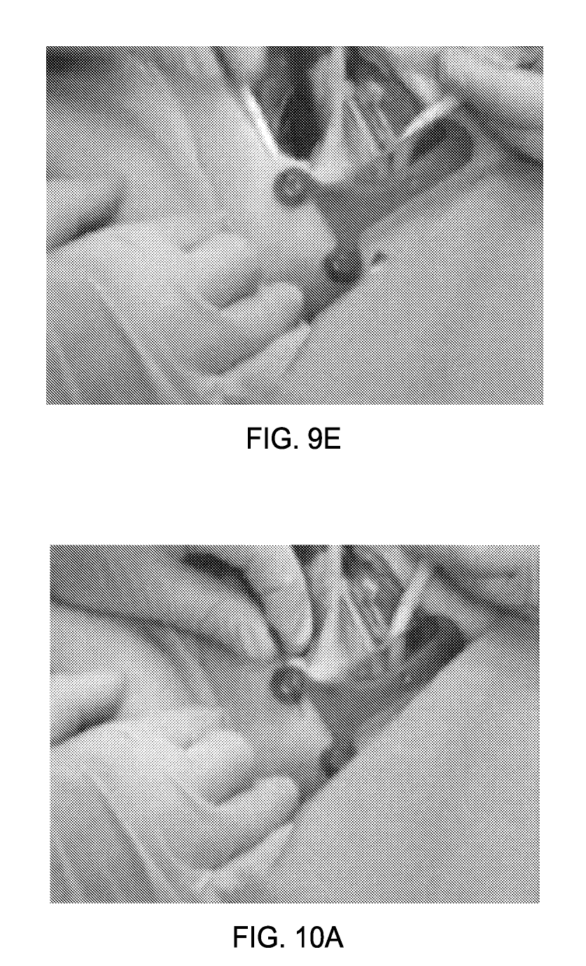

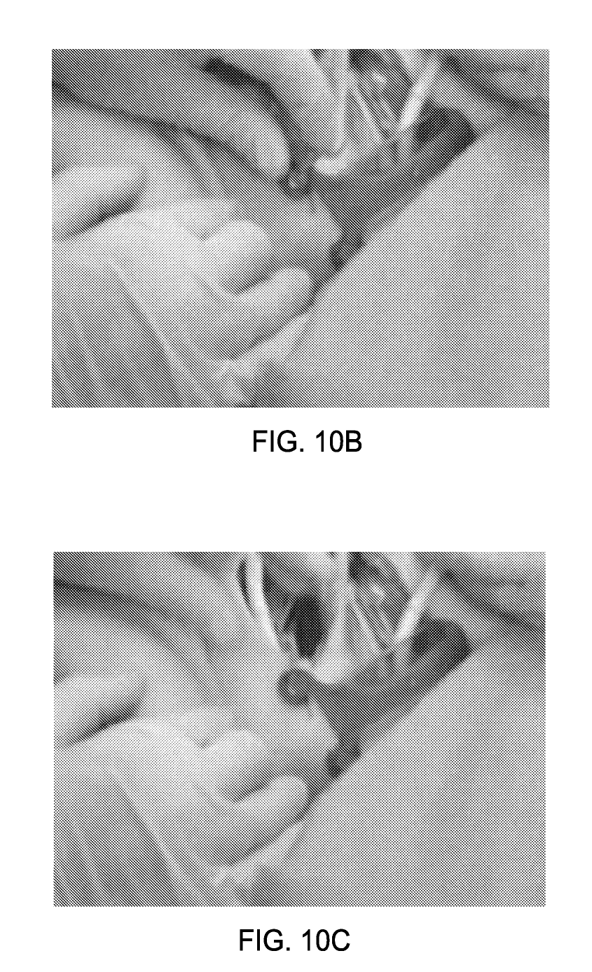

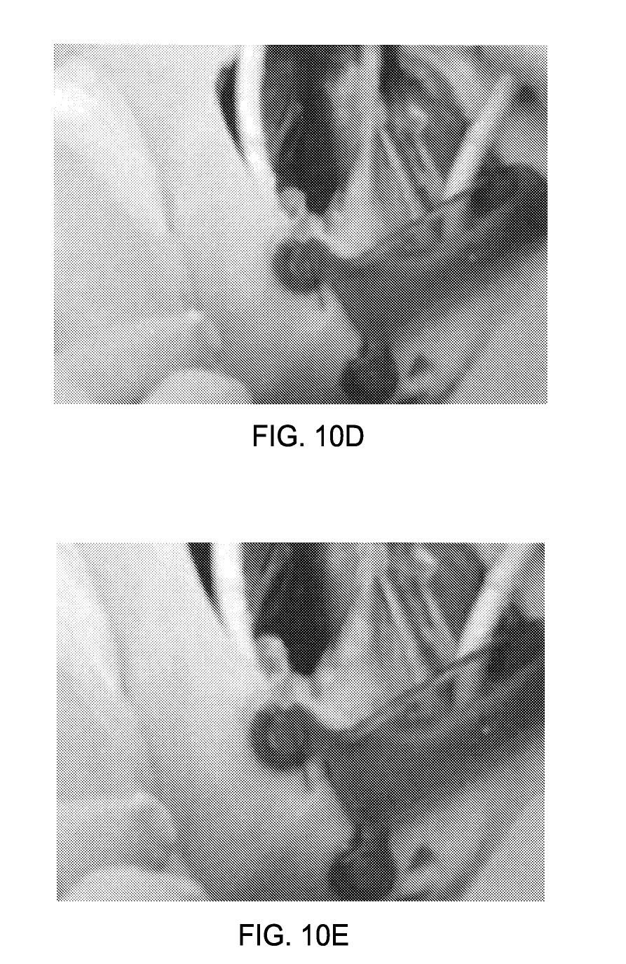

The present example demonstrates use of an illustrative AV graft of the present invention in an in situ animal model. A series of photographs taken from the animal study, and demonstrating the steps performed below is presented in FIG. 8A to FIG. 10X.

After a pig was sacrificed, post mortem incisions were made to simulate the exposures of both the left axillary artery and left external jugular veins. The graft was tunneled in the conventional subcutaneous manner. One operator placed the magnetic detector “wand” over the skin above the implanted graft. The magnets of the device detected the stainless steel rings along a first portion of the implanted graft, and localized to it (as previously shown schematically in FIG. 3B), thereby aligning the magnets in the detector to the rings in the implant (shown schematically in FIG. 2B). A second operator placed a needle through the skin into the graft (as previously shown schematically in FIG. 2C) while a third operator injected normal saline through the graft which is observed exiting retrograde through the needle's proximal hub. This in situ study clearly demonstrated that the localized puncture placed the needle within the lumen of the graft in the desired location. Two separate operators were used (one to localize the graft, and the second to puncture the graft) to demonstrate the facility of needle puncture without using any additional tactile aid, or without having prior knowledge of the precise implantation site.

### REFERENCES

The following references, to the extent that they provide exemplary procedural or other details supplementary to those set forth herein, are each specifically incorporated herein in their entirety by express reference thereto:

U.S. Pat. No. 7,351,233, entitled “Subcutaneous vascular access port, needle and kit, and methods of using same.”

U.S. Pat. No. 7,347,843, entitled “Vascular access port with needle detector.”

U.S. Pat. No. 7,297,158, entitled “Multilayer composite vascular access graft.”

U.S. Pat. No. 7,261,705, entitled “Implantable dialysis access port.”

U.S. Pat. No. 7,252,649, entitled “Implantable vascular access device.”

U.S. Pat. No. 7,101,356, entitled “Implantable vascular access device.”

U.S. Pat. No. 7,070,591, entitled “Vascular access port with physiological sensor.”

U.S. Pat. No. 7,056,316, entitled “Valve port and method for vascular access.”

U.S. Pat. No. 7,048,729, entitled “Catheter and method of fluid removal from a body cavity.”

U.S. Pat. No. 7,025,741, entitled “Arteriovenous access valve system and process.”

U.S. Pat. No. 6,997,914, entitled “Implantable access port.”

U.S. Pat. No. 6,981,969, entitled “Orthogonal arterial catheter.”

U.S. Pat. No. 6,962,580, entitled “Vascular access port with needle detector.”

U.S. Pat. No. 6,962,577, entitled “Implantable hemodialysis access device.”

U.S. Pat. No. 6,929,631, entitled “Method and apparatus for percutaneously accessing a pressure activated implanted port.”

U.S. Pat. No. 6,726,923, entitled “Apparatus and methods for preventing or treating failure of hemodialysis vascular access and other vascular grafts.”

U.S. Pat. No. 6,726,711, entitled “Artificial blood vessel with transcutaneous access ports.”

U.S. Pat. No. 6,719,783, entitled “PTFE vascular graft and method of manufacture.”

U.S. Pat. No. 6,656,151, entitled “Vascular access devices and systems.”

U.S. Pat. No. 6,582,409, entitled “Hemodialysis and vascular access systems.”

U.S. Pat. No. 6,565,594, entitled “Tunneling device.”

U.S. Pat. No. 6,527,754, entitled “Implantable vascular access device.”

U.S. Pat. No. 6,468,252, entitled “Clamp for vascular access device.”

U.S. Pat. No. 6,352,521, entitled “Valve and sealing means for hemodialysis access device.”

U.S. Pat. No. 6,319,279, entitled “Laminated self-sealing vascular access graft.”

U.S. Pat. No. 6,261,257, entitled “Dialysis graft system with self-sealing access ports.”

U.S. Pat. No. 6,007,516, entitled “Valve port and method for vascular access.”

U.S. Pat. No. 5,944,688, entitled “Implantable hemodialysis access port assembly.”

U.S. Pat. No. 5,792,104, entitled “Dual-reservoir vascular access port.”

U.S. Pat. No. 5,741,228, entitled “Implantable access device.”

Ji et al., J. Phys. Chem. C, 111:6245-6251, 2007.

Although only several exemplary embodiments have been described in detail herein, those of ordinary skill in the relevant arts will readily appreciate that many modifications are possible in the exemplary teachings without materially departing from the novel teachings and advantages of this disclosure. Accordingly, all such modifications and alternative are intended to be included within the scope of the invention as defined in the following claims. Those of ordinary skill in the art should also realize that such modifications and equivalent devices, processes, or methods do not depart from the spirit and scope of the present disclosure, and that they may readily make various changes, substitutions, and/or alterations of the devices, methods, and processes described herein without deviating from the spirit and scope of the present disclosure.

What is claimed is:

1. A vascular access graft adapted to connect an artery to a vein in a mammal, the graft comprising:
a) a substantially tubular graft having a first lumen, which graft comprises a biocompatible material and is adapted to conduct fluid in the lumen between a first end and a second end of the graft, wherein the substantially tubular graft is anastomosed at its first end to a first artery of the mammal, and is anastomosed at its second end to a first vein of the mammal;
b) at least a first and at least a second ring disposed around at least a first portion of the substantially tubular graft, an comprising a substantially magnetic or paramagnetic material, wherein the first ring is in at least substantial proximity to a first end of a cannulation site within the graft, and the second ring is also in at least substantial proximity to at least a second end of the first cannulation site within the graft,
wherein the device is implanted entirely subcutaneously in the mammal, and is adapted and configured to be penetrated substantially into a first lumen of the first cannulation site by at least a first needle or cannula.

2. The vascular access device of claim 1, further comprising at least one arterial-side tubular septum connected at opposite ends to the graft such that the first lumen is substantially continuous.

3. The vascular access device of claim 1, further comprising at least one venous-side tubular septum connected at opposite ends to the graft such that the first lumen is substantially continuous with the lumen of the anastomosed artery and the lumen of the anastomosed vein.

4. The vascular access device of claim 1, further comprising at least one arterial-side tubular septum disposed in axial alignment with the substantially tubular graft.

5. The vascular access device of claim 1, further comprising at least one venous-side tubular septum disposed in axial alignment with the substantially tubular graft.

6. The vascular access device of claim 1, further comprising at least one septum disposed along a longitudinal axis of the substantially tubular graft.

7. The vascular access device of claim 1, further comprising a first cannulating member disposed on an arterial side of the substantially tubular graft and a second cannulating member disposed on a venous side of the substantially tubular graft.

8. The vascular access device of claim 1, wherein the substantially tubular graft comprises polytetrafluoroethylene (PTFE), expanded polytetrafluoroethylene (ePTFE), polyurethane, polypropylene, polyester, or any combination thereof.

9. The vascular access device of claim 1, wherein the substantially tubular graft is anastomosed at a first end to the artery or at a second end to the vein in an “end-to-side” fashion.

10. The vascular access device of claim 1, wherein the diameter of the lumen is about 2 mm to about 12 mm.

11. The vascular access device of claim 10, wherein the diameter of the lumen is about 4 mm to about 10 mm.

12. The vascular access device of claim 1, wherein the length of the graft is about 5 cm to about 90 cm.

13. The vascular access device of claim 12, wherein the length of the graft is about 10 cm to about 50 cm.

14. The vascular access device of claim 1, wherein the cross-sectional area of the graft lumen is about 1 mm² to about 400 mm².

15. The vascular access device of claim 14, wherein the cross-sectional area of the graft lumen is about 5 mm² to about 200 mm².

16. The vascular access device of claim 1, comprising a plurality of rings positioned longitudinally along the substantially tubular graft, wherein at least a first ring of the plurality comprises at least a first magnetic or paramagnetic material.

17. The vascular access device of claim 1, wherein the substantially magnetic or paramagnetic material comprises iron, steel, surgical-grade stainless steel, cobalt, samarium, boron, nickel, or an alloy or combination thereof.

18. The vascular access device of claim 1, further comprising at least one septum on the arterial-side of the substantially tubular graft, and at least one septum on the venous-side of the substantially tubular graft, each septum fixably attached to the arterial or venous side of the graft by a sidewall that extends beyond the outer surface of the graft such that each septum further comprises at least a first ring that contains a first magnetic or paramagnetic material that is disposed at least substantially circumferentially around the outer edge of each septum.

19. The vascular access device of claim 18, wherein the at least one septum is comprised within a first port chamber that is spliced into at least a first arterial portion of the substantially tubular graft.

20. The vascular access device of claim 18, further comprising at least a second port chamber that is spliced into at least a first venous portion of the substantially tubular graft.

21. The vascular access device of claim 20, wherein the first and the second port chambers are disposed at least substantially in axial alignment along the graft, or the first and second septa are disposed substantially along a first longitudinal axis of the first and the second port chambers.

22. The vascular access device of claim 21, wherein the first and second septa each comprises a self-sealing insert, adapted to be penetrated by a needle, catheter, or cannula, to transfer a fluid into or out of the lumen of the substantially tubular graft through one or both of the port chambers.

23. The vascular access device of claim 1, wherein the magnetic or paramagnetic material comprises a surgical-grade stainless steel selected from the group consisting of series 410 stainless steel, series 416 stainless steel, series 420 stainless steel, series 430 stainless steel, and series 440 stainless steel; iron; iron oxide; steel; aluminum; copper; titanium; cobalt; boron; samarium; nickel; or any combination or alloy thereof.

24. An implantable vascular access device comprising:
a) a substantially tubular graft of biocompatible material adapted to conduct fluid in the lumen thereof, the graft anastomosed at a first end to a first artery of a mammalian patient, and anastomosed at a second end to a first vein of the patient;
b) a first substantially tubular, biocompatible, port chamber that is in fluid communication with the tubular graft, wherein the first port chamber has at least a first septum defined substantially therein, and wherein the first septum is formed by a hole defined in the first port chamber, the hole being covered by a biocompatible, self-resealing, penetrable material;
c) at least a first magnetic or paramagnetic material disposed at least substantially around at least a first portion of the edge of the port chamber, such that the paramagnetic material at least substantially defines at least a first border of the port chamber, and the first septum therein.

25. The implantable vascular access device of claim 24, wherein the magnetic or paramagnetic material comprises a ceramic material, a nanoparticle, surgical-grade steel, a metal alloy, a superparamagnetic metal oxide, aluminum, boron, cobalt, copper, iron, neodymium, nickel, samarium, titanium, or a combination or alloy thereof.

26. The implantable vascular access device of claim 24, wherein the tubular graft further comprises a plurality of ribs, rings, or protrusions, each of which substantially extending longitudinally along the graft, and circumferentially spaced apart at a substantially consistent distance along the longitudinal axis of the graft.

27. An implantable dialysis graft, comprising:
a) a tubular graft comprising a biocompatible material that is adapted and configured to conduct fluid and extending between an artery and a vein, and,
b) at least two tubular port chambers in fluid communication with the tubular graft, the port chamber having at least one septum defined therein, the septum formed by a hole defined in the port chamber, the hole being covered by a biocompatible, self-resealing, penetrable material and defined by at least a first paramagnetic ring disposed substantially around a first portion of the port chamber;
wherein the dialysis graft is implanted entirely subcutaneously, and is adapted to be cannulated by a needle disposed through the hole in the port chamber that achieves an effective flow rate for hemodialysis, and further wherein the port chamber is localized by passing a magnet along the skin of the patient into which the graft is implanted, such that the location and placement of the port chamber is facilitated by the alignment of the magnet above the at least first paramagnetic ring on the first portion of the port chamber.

28. The implantable dialysis graft of claim 27, wherein each of the at least two tubular port chambers is attached to the graft at opposite ends such that a substantially continuous lumen is formed.

29. The implantable dialysis graft of claim 27, wherein at least one of the tubular port chambers comprises a first port chamber disposed on an arterial side of the graft and a second port chamber disposed on a venous side of the graft.

30. The implantable dialysis graft of claim 27, wherein the tubular port chamber is disposed at least substantially in axial alignment with the tubular graft.

31. The implantable dialysis graft of claim 27, wherein the at least one septum on at least one of the tubular port chambers has sidewalls that extend beyond the graft such that the septum is palpable underneath the skin after the device has been implanted.

32. A system for detecting an implanted vascular graft or device, comprising (a) the vascular device of claim 1; and (b) at least one magnet sized and dimensioned to detect the implanted vascular graft when placed in proximity to the skin of a patient into which the graft or device has been implanted.

33. The system of claim 32, comprising at least two magnets, each sized and dimensioned to detect a first septum region of the implanted vascular graft that is disposed between a first and a second magnetic band positioned circumferentially around at least a first portion of the graft.

34. The system of claim 32, wherein the at least one magnet comprises a ceramic, a lanthanoid, a paramagnetic, a superparamagnetic, a ferrimagnetic, or a ferromagnetic material selected from the group consisting of aluminum, boron, cobalt, copper, iron, neodymium, nickel, samarium, titanium, and combinations or alloys thereof.

35. The system of claim 32, wherein the at least one magnet is at least substantially cylindrical or toroidal in shape, and is at least about 0.5 cm to about 5 cm in diameter.

36. A method for identifying the placement of an implanted dialysis graft, the method comprising, implanting into an animal in need thereof, the implantable dialysis graft of claim 27, then localizing the presence of the implant by passing over the skin proximate to the implant a detector that comprises one or more magnets sized and dimensioned to correspond to, and to localize to, a first cannulation site within the graft, by the attraction of the one or more magnets within the detector to one or more regions of the device that comprise a magnetic or paramagnetic material, the one or more regions operably positioned to localize the detector above the first cannulation site within the implanted dialysis graft.

37. A method for improving the accuracy of localization and cannulation of an implanted AV graft device, comprising, 1) employing the system of claim 32 to preferentially identify the location of at least a first cannulation site within the graft that is defined by the position of a plurality of magnetic or paramagnetic rings disposed along, on, or within the graft device sufficient to define the presence of the cannulation site; and 2) puncturing the first cannulation site with a needle or cannula operably positioned over the skin above the device by the magnet-facilitated localization of the detector wand above the device.
